(19) United States
(12) Patent Application Publication
(10) Pub. No.: US 2021/0251854 A1
(43) Pub. Date: Aug. 19, 2021

(54) OIL-IN-WATER TYPE SOLID COSMETIC MATERIAL AND COSMETIC PRODUCT

(71) Applicant: TOKIWA CORPORATION, Gifu (JP)

(72) Inventors: Yuka YAMAWAKI, Saitama (JP); Natsuko ISHIDA, Saitama (JP)

(73) Assignee: TOKIWA CORPORATION, Gifu (JP)

(21) Appl. No.: 17/171,332

(22) Filed: Feb. 9, 2021

(30) Foreign Application Priority Data
Feb. 19, 2020 (JP) 2020-026402

(51) Int. Cl. A61K 8/02; A61K 8/26; A61K 8/81; A61K 8/73; A61K 8/06; A61Q 1/08; A61Q 1/12; A61Q 1/10; A45D 40/00; A45D 40/26

(52) U.S. Cl. CPC A61K 8/0254; A61K 8/26; A61K 8/8147; A61K 8/73; A61K 8/731; A45D 40/26; A61Q 1/08; A61Q 1/12; A61Q 1/10; A45D 40/0068; A61K 8/062

(57) ABSTRACT

The oil-in-water type solid cosmetic material contains a powder component, a gelling agent, a water-soluble thickener, water, and an oiling agent, wherein a plate-like powder, which has an average particle size of 10 to 200 μm and has an aspect ratio of 40 to 200, is contained, as the powder component, in an amount of 3% to 30% by mass based on a total amount of the cosmetic material; and agar and/or gellan gum is contained as the gelling agent.

### TECHNICAL FIELD

The present invention relates to an oil-in-water type solid cosmetic material and a cosmetic product.

### BACKGROUND

A cosmetic material for makeup, such as blusher, eye shadow, foundation, and lipstick, is used in a form where the cosmetic material, which is charged onto a metal tray, is accommodated in a compact container. The cosmetic material present in such a form is generally applied to the skin using an application tool.

In recent years, shapes of a container for a cosmetic material and a cosmetic material itself have been devised for the purpose of enhancing visual value, such as good appearance, and convenience.

For example, Japanese Unexamined Patent Publication No. 2015-231968 (Patent Document 1) and Japanese Unexamined Patent Publication No. 2006-182736 (Patent Document 2) propose products with a cosmetic material having a three-dimensional structure in a state of protruding from a container. The cosmetic material present in such a form can be applied directly to the skin without using an application tool.

### SUMMARY

In a case where a cosmetic material comes in contact with the skin and is applied directly thereto, the cosmetic material is required to have a feeling of comfort in use, such as a refreshing sensation or cooling sensation, on the skin, and usability such as excellent adhesion to the skin. The product for external use described in Patent Document 1 is composed of oily components, and thus becomes sticky. Although the solid cosmetic material described in Patent Document 2 has the refreshing sensation derived from aqueous components, improvements are needed to achieve both usability and durability. In other words, in order to use a cosmetic material up to its usage limit, it is necessary for the cosmetic material to have durability that can withstand continuous friction with the skin and stably maintain its three-dimensional shape so as to prevent the cosmetic material from falling out of a container due to cracks or the like during use; however, for a cosmetic material containing water, in a case where the cosmetic material is simply hardened by being blended with a solidifying agent that solidifies water, it is difficult for the cosmetic material to adhere to the skin and a feeling in use is also impaired.

An object of the present invention is to provide an oil-in-water type solid cosmetic material and a cosmetic product including the same, in which the oil-in-water type solid cosmetic material has a feeling of comfort in use, such as a refreshing sensation or cooling sensation, on the skin, has usability of excellent adhesion to the skin, and has excellent durability so that the cosmetic material can be fully used up.

In order to achieve the above-mentioned object, according to the present invention, there is provided an oil-in-water type solid cosmetic material containing a powder component, a gelling agent, a water-soluble thickener, water, and an oiling agent, wherein a plate-like powder, which has an average particle size of 10 to 200 μm and has an aspect ratio of 40 to 200, is contained, as the powder component, in an amount of 3% to 30% by mass based on a total amount of the cosmetic material; and agar and/or gellan gum is contained as the gelling agent.

The oil-in-water type solid cosmetic material of the present invention can be molded into a desired shape, and the resulting molded body has a feeling of comfort in use, such as a refreshing sensation or cooling sensation, on the skin, has usability of excellent adhesion to the skin, and has excellent durability so that the cosmetic material can be fully used up. Further, in addition to the above-mentioned characteristics, the oil-in-water type solid cosmetic material of the present invention can also have smoothness so that the cosmetic material can be applied smoothly.

The plate-like powder may be at least one powder selected from the group consisting of synthetic fluorphlogopite, natural mica, metal oxide-coated mica, and metal oxide-coated glass powder.

In the oil-in-water type solid cosmetic material of the present invention, at least one selected from the group consisting of polyacrylic acid amide copolymers, xanthan gums, and cellulose derivatives may be contained, as the water-soluble thickener, in an amount of 0.3% to 2.5% by mass based on a total amount of the cosmetic material.

In the oil-in-water type solid cosmetic material of the present invention, from the viewpoint that usability such as smoothness and adhesion to the skin is further increased, a sum of contents of the agar and the gellan gum may be 0.5% to 4% by mass based on a total amount of the cosmetic material.

In the oil-in-water type solid cosmetic material of the present invention, a content of the water is preferably equal to or higher than a content of the powder component. In this case, a fresh feeling in use is easily obtained, and the cosmetic material is easily charged into a predetermined mold.

The oil-in-water type solid cosmetic material of the present invention may be molded three-dimensionally to be applied to skin by direct contact. The oil-in-water type solid cosmetic material of the present invention may be a molded body for being applied to skin by direct contact. The molded body may have a convex portion having a dome shape, a pyramid shape, a diamond cut shape, or a columnar shape.

In addition, according to the present invention, there is provided a cosmetic product, including a container and the oil-in-water type solid cosmetic material according to the present invention which is accommodated in the container, wherein the container has a container main body for holding the oil-in-water type solid cosmetic material, and the oil-in-water type solid cosmetic material is held in the container main body in a state of protruding therefrom; or the container has a container main body for receiving the oil-in-water type solid cosmetic material, and the container main body has a holding member that holds the oil-in-water type solid cosmetic material and allows the oil-in-water type solid cosmetic material to protrude from the container main body.

The cosmetic product of the present invention can be applied directly to the skin without using an application tool, can have a feeling in use, which is a fresh and refreshing feeling, and usability of excellent adhesion to the skin, and can be fully used up.

According to the present invention, it is possible to provide an oil-in-water type solid cosmetic material and a cosmetic product including the same, in which the oil-in-water type solid cosmetic material has a feeling of comfort in use, such as a refreshing sensation or cooling sensation, on the skin, has usability of excellent adhesion to the skin, and has excellent durability so that the cosmetic material can be fully used up.

### BRIEF DESCRIPTION OF THE DRAWINGS

FIG. 1 illustrates a vertical cross-sectional view showing a cosmetic product that includes an oil-in-water type solid cosmetic material according to an embodiment of the present invention.

FIG. 2 illustrates a perspective view showing a state in which a top lid of the cosmetic product of FIG. 1 is removed.

FIG. 3 illustrates a perspective view showing another three-dimensional shape of the oil-in-water type solid cosmetic material.

FIG. 4 illustrates a plan perspective view showing a container main body extracted from the views in FIGS. 1 and 2.

FIG. 5 illustrates a bottom perspective view of the container main body illustrated in FIG. 4.

FIG. 6 illustrates a state explanatory view showing a production method in which the oil-in-water type solid cosmetic material is charged into a molding space defined between a mold and a container main body.

### DETAILED DESCRIPTION

[Oil-in-Water Type Solid Cosmetic Material]

The oil-in-water type solid cosmetic material of the present embodiment contains (A) a powder component (which may be referred to as component (A)), (B) a gelling agent (which may be referred to as component (B)), and (C) a water-soluble thickener (which may be referred to as component (C)), (D) water (which may be referred to as component (D)), and (E) an oiling agent (which may be referred to as component (E)).

<(A) Powder Component>

For the powder component, known powders used in cosmetic materials may be blended with each other, and examples thereof include extender powders and coloring pigments. The powder can be used without particular limitation in terms of shapes such as spherical, plate-like, and needle-like; particle sizes such as aerosol, micro-particle, and pigment-grade; particle structures such as porous and non-porous; and the like. For the component (A), such powders may be used alone, or two or more thereof may be used in combination.

For the component (A), the oil-in-water type solid cosmetic material of the present embodiment may contain a plate-like powder (which is hereinafter also referred to as component (A-1)) having an average particle size of 10 to 200 μm and an aspect ratio of 40 to 200.

The average particle size of the plate-like powder refers to a median (50%) diameter measured by laser diffraction. In addition, the aspect ratio refers to average particle size/average thickness. The thickness is measured by observation under an electron microscope, and the average thickness is an average value obtained by dividing a sum of the measured thicknesses of the plate-like powder by the number of measurements.

For the component (A-1), from the viewpoint of durability, the oil-in-water type solid cosmetic material of the present embodiment may contain a plate-like powder having an average particle size of 10 to 200 μm, or a plate-like powder having an average particle size of 15 to 150 μm.

For the component (A-1), from the viewpoint of durability, the oil-in-water type solid cosmetic material of the present embodiment may contain a plate-like powder having an aspect ratio of 40 to 150, or a plate-like powder having an aspect ratio of 60 to 150.

For the component (A-1), synthetic fluorphlogopite (synthetic mica), natural mica, talc, sericite, barium sulfate, boron nitride, mica titanium, glass powder, PET powder, alumina, or the like may be used. The component (A-1) may be coated with a metal oxide. In this case, metal oxide coating-induced characteristics (for example, pearly feeling, coloring, and the like) can be imparted to the oil-in-water type solid cosmetic material. As the metal oxide, one of titanium oxide, iron oxide, chromium oxide, tin oxide, and alumina oxide may be used alone, or two or more thereof may be used in combination. Among these, from the viewpoint of dispersibility in an aqueous phase and shape retention, it may use at least one powder selected from the group consisting of synthetic fluorphlogopite, natural mica, metal oxide-coated mica, and metal oxide-coated glass powder; and from the viewpoint of achieving durability and usability at higher levels, it may use at least one powder selected from the group consisting of synthetic fluorphlogopite, natural mica, and metal oxide-coated mica.

The component (A-1) may be surface-treated as long as the surface treatment does not interfere with the dispersibility in an aqueous phase. Examples of the surface treatment include treatments with metal soaps, silicone compounds, fluorine compounds, surfactants, amino acid compounds, and the like.

From the viewpoint of facilitating charging and forming a molded body having both usability and durability, a content of the component (A-1) in the oil-in-water type solid cosmetic material may be 3% to 30% by mass based on a total amount of the cosmetic material; and from the viewpoint of achieving durability, feeling in use, and charging properties at higher levels, the content the component (A-1) may be 4% to 30% by mass, 5% to 30% by mass, 7% to 25% by mass, or 10% to 20% by mass, based on a total amount of the cosmetic material.

The oil-in-water type solid cosmetic material of the present embodiment may contain a plate-like powder other than the component (A-1) and a powder component other than the plate-like powder. Examples of such powders may include inorganic coloring pigments such as black iron oxide, red iron oxide, yellow iron oxide, cobalt oxide, chromium oxide, Ultramarine Blue, Iron Blue, titanium oxide, and zinc oxide; organic coloring pigments such as Red No. 228, Red No. 226, Blue No. 404, Red No. 202, and Yellow No. 4 Aluminum Lake; pearl pigments such as mica titanium, fish scale foil, and bismuth oxychloride; natural pigments such as carmine and carthamus; extender pigments such as glass powder, silicic anhydride, aluminum silicate, magnesium silicate, aluminum magnesium silicate, mica, synthetic mica, synthetic sericite, sericite, talc, kaolin, silicon carbide, and barium sulfate; and spherical powders such as crosslinked polymethylmethacrylate and silicone elastomer. These powders may be surface-treated for the purpose of improving usability, dispersibility, and the like. Examples of the surface treatment include treatments with metal soaps, silicone compounds, fluorine compounds, surfactants, amino acid compounds, and the like.

From the viewpoint of durability, usability, and charging properties, a content of the component (A) in the oil-in-water type solid cosmetic material may be 3% to 40% by mass, or 3% to 35% by mass, based on a total amount of the cosmetic material.

<(B) Gelling Agent>

For the gelling agent, known gelling agents used in cosmetic materials may be blended with each other, and it is possible to use a gelling agent having an effect of solidifying an aqueous component containing water. As the component (B), such gelling agents may be used alone, or two or more thereof may be used in combination.

For the component (B), the oil-in-water type solid cosmetic material of the present embodiment may contain agar and/or gellan gum.

From the viewpoint of further increasing durability, and usability such as smooth applicability and adhesion, a total content of the component (B) in the oil-in-water type solid cosmetic material may be 0.5% to 4% by mass, 0.5% to 3.5% by mass, or 1% to 3.0% by mass, based on a total amount of the cosmetic material.

<(C) Water-Soluble Thickener>

For the water-soluble thickener, those other than the component (B) may be used, and examples thereof include cellulosic thickeners, polysaccharide thickeners, clay minerals, and acrylic acid copolymer thickeners. For the component (C), such thickeners may be used alone, or two or more thereof may be used in combination.

Examples of the cellulosic thickener include cellulose derivatives such as methyl cellulose, ethyl cellulose, cellulose gum, hydroxyethyl cellulose, hydroxypropyl cellulose, and hydroxypropyl methyl cellulose.

Examples of the polysaccharide thickener include pectin, guar gum, xanthan gum, carrageenan, gellan gum, and gum arabic.

Examples of the acrylic acid copolymer thickener include carboxyvinyl polymers, alkyl-modified carboxyvinyl polymers, and polyacrylic acid amide copolymers. Among these, it may use polyacrylic acid amide copolymers may be used, or polyacrylic acid amide copolymers containing acryloyldimethyltaurate as a copolymerizablecomponent.

Examples of the polyacrylic acid amide copolymer containing acryloyldimethyl taurate as a copolymerizable component include hydroxyethyl acrylate/sodium acryloyldimethyl taurate copolymer, sodium acrylate/sodium acryloyldimethyl taurate copolymer, ammonium acryloyldimethyl taurate/VP copolymer, and ammonium acryloyldimethyltaurate/beheneth-25 methacrylate crosspolymer. Among these, hydroxyethyl acrylate/sodium acryloyldimethyl taurate copolymer may be used. In addition, a commercially available product containing a polyacrylic acid amide copolymer may be used.

For the component (C), the oil-in-water type solid cosmetic material of the present embodiment may contain at least one selected from the group consisting of polyacrylic acid amide copolymers, xanthan gums, and cellulose derivatives.

From the viewpoints of durability, smooth applicability, and adhesion, a total content of the component (C) in the oil-in-water type solid cosmetic material may be 0.3% to 2.5% by mass, 0.5% to 2.5% by mass, 0.5% to 2% by mass, 0.8% to 1.5% by mass, or 1.1% to 1.5% by mass, based on a total amount of the cosmetic material.

In the oil-in-water type solid cosmetic material of the present embodiment, a ratio of the component (C) to the component (A-1)([content of component (C)]/[content of component (A-1)]) may be 0.01 to 0.7. From the viewpoint of durability, smooth applicability, and adhesion, the ratio may be 0.02 to 0.5, or 0.05 to 0.3.

<(D) Water>

From the viewpoint of imparting the refreshing sensation and facilitating charging of the cosmetic material into a predetermined mold, a content of the component (D) in the oil-in-water type solid cosmetic material of the present embodiment may be equal to or higher than the content of the component (A).

In addition, from the viewpoint of charging properties and durability, the content of the component (D) may be 30% to 70% by mass, or 40% to 65% by mass, based on a total amount of the cosmetic material.

<(E) Oiling Agent>

Examples of the component (E) include fats and oils, waxes, hydrocarbons, ester oils, higher alcohols, higher fatty acids, silicone oils, and oil-soluble ultraviolet absorbers. For the component (E), such oiling agents may be used alone, or two or more thereof may be used in combination.

Examples of the fat and oil include solid fats and oils such as hardened castor oil, hydrogenated jojoba oil, palm oil, and Japanese wax, and liquid oils and fats such as jojoba seed oil, sunflower seed oil, olive oil, castor oil, macadamia nut oil, camellia oil, rapeseed oil, linseed oil, and triglycerin.

Examples of the waxes include beeswax, carnauba wax, candelilla wax, rice bran wax, sunflower seed wax, bran wax, whale wax, and lanolin.

Examples of the hydrocarbon include polyethylene wax, microcrystalline wax, Fischer-Tropsch wax, ceresin, petrolatum, liquid paraffin, squalane, and mineral oil.

Examples of the ester oil include glyceryl tribehenate, cholesterol fatty acid ester, diisostearyl malate, isopropyl myristate, cetyl 2-ethylhexanoate, isopropyl palmitate, ethylhexyl palmitate, glyceryl tricaprylate-caprate, neopentyl glycol dicaprate, glyceryl tri-2-ethylhexanoate, polyglyceryl triisostearate, dipentaerythrityl tetra(hydroxystearate/isostearate), neopentyl glycol di-2-ethylhexanoate, triethylhexanoin, and dimer acid ester.

Examples of the higher alcohol include stearyl alcohol, behenyl alcohol, cetyl alcohol, cetostearyl alcohol, oleyl alcohol, octyldodecanol, and isostearyl alcohol.

Examples of the higher fatty acid include stearic acid, oleic acid, myristic acid, palmitic acid, isostearic acid, behenic acid, linoleic acid, and linolenic acid.

Examples of the silicone oil include dimethylpolysiloxane, methylphenylpolysiloxane, octamethylcyclotetrasiloxane, decamethylcyclopentasiloxane, and methyltrimethicone.

From the viewpoint of usability, a total content of the component (E) in the oil-in-water type solid cosmetic material may be 0.1% to 20% by mass, 0.5% to 15% by mass, 1% to 10% by mass, or 1% to 5% by mass, based on a total amount of the cosmetic material.

<Other Components>

The oil-in-water type solid cosmetic material of the present embodiment may contain components other than those described above. For the other components, components commonly used in cosmetic products, for example, aqueous components such as moisturizers and lower alcohols, UV absorbers, defoamers, surfactants, preservatives, vitamins, beauty components, antioxidants, and fragrances may be appropriately blended with each other if necessary, as long as an effect of the present invention is not impaired.

Examples of the moisturizer include polyhydric alcohols such as 1,3-butylene glycol (BG), dipropylene glycol (DPG), glycerin, 1,2-pentanediol, sorbitol, and mannitol. These may be used alone, or two or more thereof may be used in combination.

Examples of the lower alcohol include ethanol, propyl alcohol, isopropyl alcohol, and isobutyl alcohol. These may be used alone, or two or more thereof may be used in combination.

Examples of the surfactant include anionic surfactants, cationic surfactants, amphoteric surfactants, and nonionic surfactants.

Examples of the nonionic surfactant include polyoxyalkylene alkyl ether, glycerin alkyl ether, glycerin fatty acid ester, polyglycerin fatty acid ester, sorbitan fatty acid ester, and alkylene glycol adducts of those listed immediately before, polyalkylene glycol fatty acid ester, polyglycerin-modified silicone, and polyether-modified silicone. Examples of the anionic surfactant include alkyl phosphates, polyoxyalkylene alkyl ether phosphates, sulfonates, alkyl sulfates, and polyaspartates. Examples of the cationic surfactant include alkylamine salts and alkyltrimethylammonium salts. Examples of the amphoteric surfactant include hydrogenated lecithin, carbobetaine amphoteric surfactants, sulfobetaine amphoteric surfactants, and amino acid amphoteric surfactants.

The surfactants may be used alone, or two or more thereof may be used in combination.

A content of the surfactant in the oil-in-water type solid cosmetic material of the present embodiment may be 1% by mass or lower, 0.7% by mass or lower, or 0.5% by mass or lower, based on a total amount of the cosmetic material.

From the viewpoint of durability and usability, the oil-in-water type solid cosmetic material of the present embodiment may have a bulk hardness of 0.2 to 1.5 N, or a bulk hardness of 0.1 to 0.5 N. The bulk hardness of the cosmetic material can be determined by the following measurement method.

<Measurement Method of Bulk Hardness>

First, a cosmetic material melted by heating is charged into a screw cup having a capacity of 30 mL, and left to stand at 25° C. overnight, to prepare a sample for measurement. For this sample for measurement, the hardness is measured using the FUDOH rheometer RT-2002D⋅D (manufactured by RHEOTECH) at 25° C. with a 3ϕ columnar pressure-sensitive shaft, a needle insertion speed of 6 cm/min, and a needle insertion depth of 10 mm.

The oil-in-water type solid cosmetic material of the present embodiment may be molded three-dimensionally to be applied to skin by direct contact. The oil-in-water type solid cosmetic material of the present embodiment may be a molded body for being applied to skin by direct contact. A shape of the three-dimensional molding may be a hemispherical shape or a shape having a convex portion similar to a hemispherical shape. By holding the cosmetic material in a container main body so that such a convex portion protrudes from the container main body, it is possible to cause the convex portion to come in contact with the skin and be applied directly thereto.

The convex portion may be molded into a dome shape, a pyramid shape, a diamond cut shape, or a columnar shape, or may be designed so as to be provided with a part where a height difference is intentionally created, like a petal shape.

The oil-in-water type solid cosmetic material of the present embodiment may be monochromatic or multichromatic. In a case where the cosmetic material is multichromatic, for example, parts of different colors may be integrally molded.

Examples of uses of the oil-in-water type solid cosmetic material of the present embodiment include cosmetic materials for makeup, such as blusher, eye shadow, foundation, lipstick, concealer, highlighter, makeup base, and eyebow pencil; cosmetic materials for skin care, such as beauty liquid and moisturizer; cosmetic materials for body, such as sunscreen and solid perfume; and cosmetic materials for hair styling, such as temporary hair dying agent.

[Method for Producing Oil-in-Water Type Solid Cosmetic Material]

In a method for producing the oil-in-water type solid cosmetic material according to the present embodiment, for example, it is possible to obtain the cosmetic material by mixing the above-described components (A), (B), (C), (E), and other components with water, which is the component (D), charging this mixture into a predetermined container main body, and performing solidification.

The mixing procedure may be carried out, for example, by preparing an aqueous phase containing the components (A), (B), (C), and (D), and if necessary, a surfactant, and an oil phase containing the component (E) and the other components, respectively, adding the oil phase to the aqueous phase, and performing stirring so that the mixture is emulsified. A temperature at the time of preparing the aqueous phase may be a temperature at which dissolution of the components (B) and (C) occurs. A temperature at the time of preparing the oil phase may be a temperature at which dissolution of the component (E) occurs.

A temperature at the time of charging the oil-in-water type solid cosmetic material may be 60° C. to 90° C., and may be adjusted so that a viscosity at the time of charging the oil-in-water type solid cosmetic material is 1,000 to 50,000 mPa·s. The viscosity at charging is a numerical value measured with a B-type viscometer at a predetermined temperature at charging.

[Cosmetic Product]

The cosmetic product according to the present invention includes a container and the above-described oil-in-water type solid cosmetic material according to the present embodiment which is accommodated in the container, wherein the container has a container main body for holding the oil-in-water type solid cosmetic material, and the oil-in-water type solid cosmetic material is held in the container main body in a state of protruding therefrom; or the container has a container main body for receiving the oil-in-water type solid cosmetic material, and the container main body has a holding member that holds the oil-in-water type solid cosmetic material and allows the oil-in-water type solid cosmetic material to protrude from the container main body.

For the container having a container main body for receiving the oil-in-water type solid cosmetic material, in which the container main body has a holding member that holds the oil-in-water type solid cosmetic material and allows the oil-in-water type solid cosmetic material to protrude from the container main body, for example, it is possible to use a common container used for lipsticks or the like, such as a container having a mechanism that allows a cosmetic material with a stick-like shape to move forward and backward.

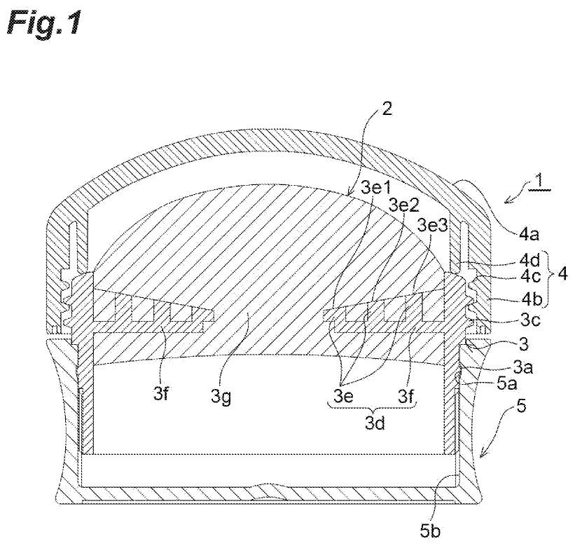

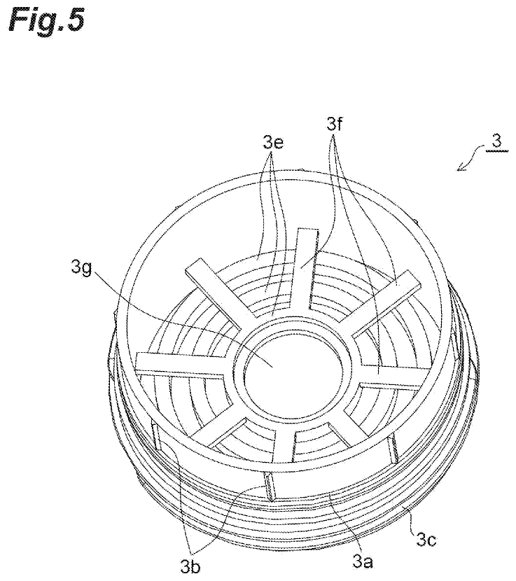

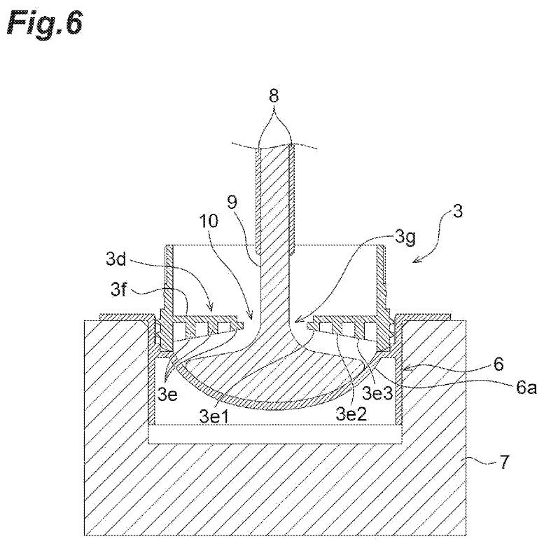

In the present embodiment, from the viewpoints that a cosmetic material can be used without having to cause the cosmetic material to move forward and excellent usability is achieved, it is preferable that the oil-in-water type solid cosmetic material be held in a container main body in a state of protruding from the container main body. Hereinafter, such an embodiment will be described with reference to FIGS. 1 to 6. FIGS. 1 to 5 illustrate views showing cosmetic products according to the present embodiment, and FIG. 6 illustrates a view showing a method for producing an oil-in-water type solid cosmetic material which forms a convex shape and is held in a container. The cosmetic product is applicable to, for example, cosmetic materials for makeup, such as blusher, eye shadow, foundation, lipstick, concealer, highlighter, makeup base, and eyebrow pencil; cosmetic materials for skin care, such as beauty liquid and moisturizer; cosmetic materials for body, such as sunscreen and solid perfume; and cosmetic materials for hair styling, such as temporary hair dying agent.

As illustrated in FIG. 1, the cosmetic product of the present embodiment includes a container 1 that consists mainly of an oil-in-water type solid cosmetic material 2 and a container main body 3 as a middle plate for holding the oil-in-water type solid cosmetic material 2, in which the container 1 includes a top lid 4 that covers an upper part of the container main body 3 so as to cover the oil-in-water type solid cosmetic material 2, and a bottom lid 5 that covers a lower part of the container main body 3. The container main body 3, the top lid 4, and the bottom lid 5 are formed of, for example, resin or the like.

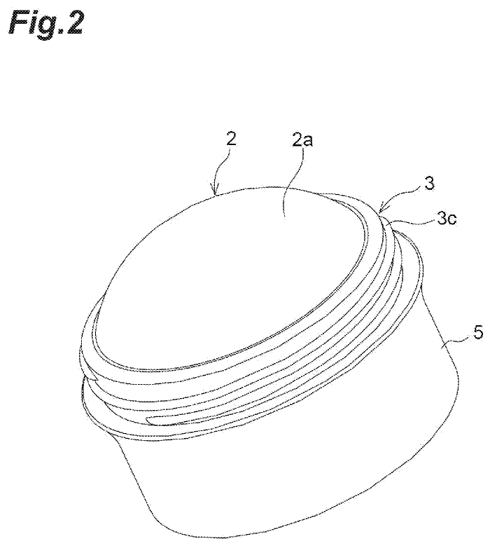

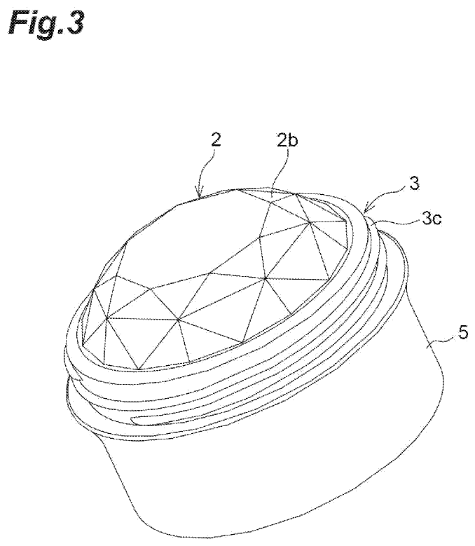

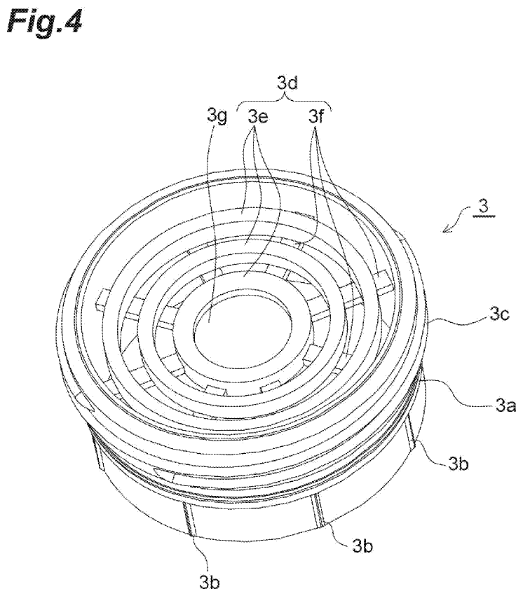

As illustrated in FIGS. 1, 2, 4, and 5, the container main body 3 has a cylindrical shape. As illustrated in FIGS. 1 and 4, a holding member 3d provided on an inner peripheral side is configured to be such that as illustrated in FIGS. 1 and 2, the holding member 3d holds the oil-in-water type solid cosmetic material 2 having a substantially hemispherical convex portion 2a that protrudes upward so as to fill a cylindrical space. FIG. 3 illustrates an example of another three-dimensional shape of the oil-in-water type solid cosmetic material, in which a convex portion 2b is molded into a diamond cut shape. A shape of the convex portion is not limited to these, and may be appropriately changed into the above-mentioned shape or the like.

The container main body 3 is provided with a male screw 3c for detachably mounting the top lid 4 along an upper outer peripheral surface, as illustrated in FIGS. 1 and 4; is provided, on an open end side of a lower outer peripheral surface, with a plurality of protruding shapes 3b for restraining relative rotation with the bottom lid 5 at equidistant positions in a circumferential direction, as illustrated in FIG. 4; and is provided, at a position above the protruding shape 3b, with an annular convex portion 3a for restraining vertical movement of the bottom lid 5, as illustrated in FIGS. 1 and 4.

As illustrated in FIG. 1, the top lid 4 having a bottomed cylindrical shape includes a dome-shaped covering portion 4a that faces the oil-in-water type solid cosmetic material 2 and covers the oil-in-water type solid cosmetic material 2 from above, and an annular peripheral wall portion 4b connected to a peripheral edge of the covering portion 4a; and the top lid 4 is detachably mounted on the container main body 3 in such a manner that a female screw 4c, which is externally coupled to an upper part of the container main body 3 and formed on an inner peripheral surface of the peripheral wall portion 4b, is screwed into the male screw 3c of the container main body 3.

Regarding screwing between the male screw 3c of the container main body 3 and the female screw 4c of the top lid 4, an annular convex portion 4d protruding near the peripheral wall portion 4b on an inner surface of the covering portion 4a of the top lid 4 abuts on an upper end surface of the container main body 3 so that overtightening of the screws 3c and 4c is prevented.

The bottom lid 5, which has a bottomed cylindrical shape and is externally coupled to a lower part of the container main body 3, is fixed to the container main body 3 in both the rotational direction and the vertical direction, in such a manner that the protruding shapes 3b of the container main body 3 enter a large number of knurls Sb formed along an inner peripheral surface of the annular peripheral wall so that relative rotation with the container main body 3 is restrained, and the annular convex portion 3a of the container main body 3 engages with an annular groove portion Sa formed along an inner peripheral surface above the knurl 5b of the peripheral wall.

In the present embodiment, the holding member 3d of the container main body 3 for holding the oil-in-water type solid cosmetic material 2 is configured to include an annular holding member 3e and a radial holding member 3f. However, a shape of the holding member may be appropriately changed.

The annular holding member 3e has a rectangular cross-sectional shape as illustrated in FIG. 1, and a plurality (three in the drawing) thereof are arranged concentrically as illustrated in FIGS. 1 and 4. As illustrated in FIG. 1, in these adjacent annular holding members 3e and 3e, the outer annular holding member is raised upward and specifically, an upper surface 3e2 of the middle annular holder 3e is higher than an upper surface 3e1 of the innermost annular holder 3e, and the upper surface 3e3 of the outermost annular holding member 3e is higher than the upper surface 3e2 of the middle annular holding member 3e, in which these upper surfaces 3e1, 3e2, and 3e3 are configured as inclined surfaces that are inclined at substantially the same angle and extend spaced apart from each other. Then, as illustrated in FIGS. 1, 4 and 5, an opening 3g surrounded by the innermost annular holding member 3e having the upper surface 3e1 is set as a charging port into which the oil-in-water type solid cosmetic material in a dissolved state as described later is poured.

The radial holding member 3f is connected to the innermost annular holding member 3e having the upper surface 3e1, and a plurality (eight in the drawing) thereof radially extend from the innermost annular holding member 3e and connect to an inner peripheral surface of the container main body 3. These radial holding members 3f are connected to the annular holding members 3e so that upper parts of the radial holders 3f are located on positions lower than upper parts of the annular holding members 3e, thereby causing the annular holding members 3e to be supported on the container main body 3.

Then, as illustrated in FIG. 1, the holding member 3d including these annular and radial holding members 3e and 3f is configured to be such that the holding member 3d is in a state of being covered with the oil-in-water type solid cosmetic material 2, thereby holding the oil-in-water type solid cosmetic material 2.

Next, production of the oil-in-water type solid cosmetic material 2 held in the holding member 3d of the container main body 3 will be described with reference to FIG. 6.

First, a manufacturing machine provided with a holding body 7 and a charging nozzle 8, which is located above the holding body 7 and ejects an oil-in-water type solid cosmetic material in a dissolved state, is prepared.

Next, the container main body 3 is fitted upside down in a lipstick mold (molding mold) 6 provided with a dome-shaped concave portion 6a so that the lipstick mold 6 is set in the holding body 7. The lipstick mold 6 is made of, for example, aluminum, resin, rubber, or the like.

Next, the oil-in-water type solid cosmetic material 9 in a heat-dissolved state is poured from the charging nozzle 8 into the container main body 3 through the opening 3g.

By doing so, the oil-in-water type solid cosmetic material in a dissolved state is charged into the molding space 10 defined between the lipstick mold 6 and the container main body 3.

Charging of the oil-in-water type solid cosmetic material is carried out until the oil-in-water type solid cosmetic material covers the holding member 3d. The oil-in-water type solid cosmetic material may be monochromatic or multichromatic. In a case where the cosmetic material is multichromatic, charging of oil-in-water type solid cosmetic materials of different colors may be done separately or all at once. In addition, in a case where the holding body 7 is a rotating body and the charging nozzle 8 includes a plurality of charging nozzles, a spiral pattern having a plural or higher number of colors can be formed. That is, as the rotating body rotates, the lipstick mold 6 and the container main body 3 rotate; and simultaneously with the start of the rotation, or when a predetermined rotation is reached, the oil-in-water type solid cosmetic materials of different colors in a heat-dissolved state are poured almost at the same time from the charging nozzles to form a spiral pattern.

Then, when the oil-in-water type solid cosmetic material is solidified, the container main body 3 is released from the lipstick mold 6. In such a manner, a substantially hemispherical oil-in-water type solid cosmetic material 2 held in the holding member 3d of the container main body 3 is obtained.

A temperature at the time of charging the oil-in-water type solid cosmetic material may be set under the above-described conditions.

Then, when the container main body 3, in which the oil-in-water type solid cosmetic material 2 is held, is obtained, the top lid 4 is detachably mounted on the container main body 3 in such a manner that the top lid 4 is externally coupled from its open end side to an upper part of the container main body 3 so that the top lid 4 and the container main body 3 are rotated relative to each other and thus the female screw 4c of the top lid 4 is screwed into the male screw 3c of the container main body 3. Next, the bottom lid 5 is fixed to the container main body 3 in such a manner that the bottom lid 5 is externally coupled from its open end side to a lower part of the container main body 3, on which the top lid 4 is mounted, so that the protruding shape 3b of the container main body 3 enters the knurl 5b of the bottom lid 5 and the annular groove portion 5a of the bottom lid 5 engages with the annular convex portion 3a of the container main body 3. In such a manner, the cosmetic product illustrated in FIG. 1 is obtained.

According to the cosmetic product having such a configuration, the oil-in-water type solid cosmetic material 2 having a substantially hemispherical convex portion is firmly held by the annular holding member 3e in such a manner that the oil-in-water type solid cosmetic material 2 is interposed between the plurality of annular holding members 3e. Thus, the oil-in-water solid cosmetic material 2 is prevented from falling off from the container main body 3 due to an impact such as dropping. In addition, the oil-in-water type solid cosmetic material 2 is further firmly held by the radial holding member 3f in such a manner that the oil-in-water type solid cosmetic material 2 is interposed between the plurality of radial holding members 3f. Thus, the oil-in-water solid cosmetic material 2 is further prevented from falling off from the container main body 3.

Next, in a case of applying the oil-in-water type solid cosmetic material 2, first, the top lid 4 and the bottom lid 5 are grasped by hands and rotated relative to each other, to unscrew the female screw 4c of the top lid 4 from the male screw 3c of the container main body 3. Then, the top lid 4 is removed from the container main body 3, and thus the container main body 3 is in a state as illustrated in FIG. 2. In such a state, the oil-in-water type solid cosmetic material 2, which protrudes from the container main body 3 in a substantially hemispherical shape, may be applied. Such a cosmetic product can be used without having to cause the cosmetic material to move forward, and thus has excellent convenience.

As such an application proceeds, the oil-in-water type solid cosmetic material 2 is consumed and decreased to reach the vicinity of the annular holding member 3e that is close to a consumption limit. However, the oil-in-water type solid cosmetic material according to the present embodiment has excellent shape retention, and thus the oil-in-water type solid cosmetic material can be fully used up until its consumption limit while having a feeling in use, which is a fresh and refreshing feeling, and usability of excellent adhesion to the skin.

The annular groove portion 5a of the bottom lid 5 and the annular convex portion 3a of the container main body 3 may be removably (detachably) engaged with each other. With this configuration, in a case where there is a need to replace the oil-in-water type solid cosmetic material 2 as the oil-in-water type solid cosmetic material 2 is used up to its consumption limit, the top lid 4 is relatively rotated with respect to the container main body 3 to be unscrewed and removed therefrom, and the bottom lid 5 is disengaged and removed from the container main body 3. In such a state, it is possible to replace the used-up container main body 3 with a replacement container main body 3, in which a new oil-in-water type solid cosmetic material 2 is held.

### EXAMPLES

Hereinafter, the present invention will be described in more detail by way of examples. However, the technical scope of the present invention is not limited by these examples. Each of numerical values in tables indicates a content (% by mass) based on a total amount of a cosmetic base material.

Prior to describing the examples, evaluation methods adopted in the respective examples will be described.

<Hardness of Cosmetic Material>

A bulk hardness of a cosmetic material was determined by the following measurement method.

First, a cosmetic material melted by heating was charged into a screw cup having a capacity of 30 mL, and left to stand at 25° C. overnight, to prepare a sample for measurement. For this sample for measurement, the hardness was measured using the FUDOH rheometer RT-2002D⋅D (manufactured by RHEOTECH) at 25° C. with a 3ϕ columnar pressure-sensitive shaft, a needle insertion speed of 6 cm/min, and a needle insertion depth of 10 mm.

<Evaluation of Durability of Dome Shape>

A cosmetic evaluation expert panel of 10 people was asked to apply, to an inside of the arm, each of the oil-in-water type solid cosmetic materials of the examples and comparative examples. For each sample, a score was given by performing a five-grade evaluation according to the following evaluation criteria. Further, the scores given by all panel members were averaged, and the average score was used to make a determination according to the following determination criteria.

[Score: Evaluation Criteria]

5 points: All can be completely used

4 points: All can be almost completely used

3 points: ¾ or higher can be used

2 points: ½ or higher can be used

1 point: ¼ or higher can be used

[Determination Criteria (Average Score)]

A: Equal to or higher than 4

B: Equal to or higher than 3 and lower than 4

C: Equal to or higher than 2 and lower than 3

D: Lower than 2

<Evaluation of Usability>

A cosmetic evaluation expert panel of 10 people was asked to apply, to the cheeks, each of the oil-in-water type solid cosmetic materials of the examples and comparative examples, and to evaluate usability by comprehensively considering smooth applicability, a feeling of comfort in use such as a refreshing sensation or cooling sensation, on the skin, and adhesion to the skin. Regarding evaluation of the usability, for each sample, a score was given by performing a five-grade evaluation according to the following evaluation criteria. Further, the scores given by all panel members were averaged, and the average score was used to make a determination according to the following determination criteria.

[Score: Evaluation Criteria]

5 points: Very good

4 points: Good

3 points: Normal

2 points: Slightly poor

1 point: Poor

[Determination Criteria (Average Score)]

A: Equal to or higher than 4

B: Equal to or higher than 3 and lower than 4

C: Equal to or higher than 2 and lower than 3

D: Lower than 2

### Examples 1 to 24 and Comparative Examples 1 to 10

An oil-in-water type solid cosmetic material (blusher) having each composition (% by mass) shown in Tables 1 to 4 was prepared by the following production method and evaluated as described above. The results are together shown in Tables 1 to 4.

<Production Method>

First, aqueous phase components were dispersed in purified water, and this dispersion was heated and dissolved at 90° C. or higher, to prepare an aqueous phase portion. Subsequently, to the aqueous phase portion was added an oil phase portion, in which oil phase components were uniformly mixed, and the mixture was sufficiently stirred and emulsified. The obtained solution was charged into a predetermined mold at 60° C. or higher and cooled to obtain an oil-in-water type solid cosmetic material having a shape similar to that illustrated in FIG. 1. Then, the obtained solid cosmetic material was transferred into a predetermined container that causes a convex portion of the oil-in-water type solid cosmetic material to be exposed, and this was used as a sample.

TABLE 1

| | | | | | | | | | | Compar- | Compar- |
|---|---|---|---|---|---|---|---|---|---|---|---|
| | | | Example | Example | Example | Example | Example | Example | Example | ative | ative |
| | | | 1 | 2 | 3 | 4 | 5 | 6 | 7 | Example 1 | Example 2 |
| Aqueous | Purified water | | to 100 | to 100 | to 100 | to 100 | to 100 | to 100 | to 100 | to 100 | to 100 |
| phase | (A-1) | Synthetic | 10.5 | — | — | 5.0 | — | — | — | — | — |
| | | fluorphlogopite 1 | | | | | | | | | |
| | | Synthetic | — | 10.5 | — | — | 5.0 | — | — | — | — |
| | | fluorphlogopite 2 | | | | | | | | | |
| | | Synthetic | — | — | 10.5 | 5.5 | 5.5 | 10.5 | 10.5 | — | — |
| | | fluorphlogopite 3 | | | | | | | | | |
| | Synthetic fluorphlogopite 4 | | — | — | — | — | — | — | — | 10.5 | — |
| | Synthetic fluorphlogopite 5 | | — | — | — | — | — | — | — | — | 10.5 |
| | Colorant | | 1.0 | 1.0 | 1.0 | 1.0 | 1.0 | 1.0 | 1.0 | 1.0 | 1.0 |
| | (B) | Agar | 2.3 | 2.3 | 2.3 | 2.3 | 2.3 | 2.3 | 2.3 | 2.3 | 2.3 |
| | (C) | Polyacrylic acid | 1.3 | 1.3 | 1.3 | 1.3 | 1.3 | — | — | 1.3 | 1.3 |
| | | amide copolymer 1 | | | | | | | | | |
| | | Xanthan gum | — | — | — | — | — | 1.3 | — | — | — |
| | | Cellulose gum | — | — | — | — | — | — | 1.3 | — | — |

| | | | | | | | | | | |
|---|---|---|---|---|---|---|---|---|---|---|
| | BG | 7.0 | 7.0 | 7.0 | 7.0 | 7.0 | 7.0 | 7.0 | 7.0 | 7.0 |
| | DPG | 10.0 | 10.0 | 10.0 | 10.0 | 10.0 | 10.0 | 10.0 | 10.0 | 10.0 |
| | Glycerin | 8.5 | 8.5 | 8.5 | 8.5 | 8.5 | 8.5 | 8.5 | 8.5 | 8.5 |
| | Hydrogenated lecithin | 0.1 | 0.1 | 0.1 | 0.1 | 0.1 | 0.1 | 0.1 | 0.1 | 0.1 |
| | PEG-60 hydrogenated | 0.3 | 0.3 | 0.3 | 0.3 | 0.3 | 0.3 | 0.3 | 0.3 | 0.3 |
| | castor oil | | | | | | | | | |
| | Preservative | Adequate | Adequate | Adequate | Adequate | Adequate | Adequate | Adequate | Adequate | Adequate |
| | | amount | amount | amount | amount | amount | amount | amount | amount | amount |
| Oil phase | Neopentyl glycol | 1.1 | 1.1 | 1.1 | 1.1 | 1.1 | 1.1 | 1.1 | 1.1 | 1.1 |
| | diethylhexanoate | | | | | | | | | |
| | Squalane | 0.5 | 0.5 | 0.5 | 0.5 | 0.5 | 0.5 | 0.5 | 0.5 | 0.5 |
| | Defoamer | Adequate | Adequate | Adequate | Adequate | Adequate | Adequate | Adequate | Adequate | Adequate |
| | | amount | amount | amount | amount | amount | amount | amount | amount | amount |
| | Antioxidant | Adequate | Adequate | Adequate | Adequate | Adequate | Adequate | Adequate | Adequate | Adequate |
| | | amount | amount | amount | amount | amount | amount | amount | amount | amount |
| Evaluation | Hardness (N) | 0.41 | 0.41 | 0.43 | 0.42 | 0.42 | 0.30 | 0.45 | 0.39 | 0.40 |
| | Durability (score) | B (3.0) | A (4.3) | A (5.0) | A (5.0) | A (4.8) | B (3.5) | A (4.8) | D (1.0) | D (1.0) |
| | Usability | A | A | A | A | A | B | A | A | A |

TABLE 2

| | | | Example | Example | Example | Example | Example | Comparative |
|---|---|---|---|---|---|---|---|---|
| | | | 8 | 9 | 10 | 11 | 12 | Example 3 |
| Aqueous | Purified water | | to 100 | to 100 | to 100 | to 100 | to 100 | to 100 |
| phase | (A-1) | Natural mica 1 | 10.5 | — | — | — | — | — |
| | | Natural mica 2 | — | 10.5 | — | — | — | — |
| | | Mica titanium | — | — | 10.5 | — | — | — |
| | | Titanium | — | — | — | 10.5 | — | — |
| | | oxide-coated | | | | | | |
| | | glass powder 1 | | | | | | |
| | | Titanium | — | — | — | — | 10.5 | — |
| | | oxide-coated | | | | | | |
| | | glass powder 2 | | | | | | |
| | Natural mica 3 | | — | — | — | — | — | 10.5 |
| | Natural mica 4 | | — | — | — | — | — | — |
| | Talc | | — | — | — | — | — | — |
| | Spherical silica | | — | — | — | — | — | — |
| | Colorant | | 1.0 | 1.0 | 1.0 | 1.0 | 1.0 | 1.0 |
| | (B) | Agar | 2.3 | 2.3 | 2.3 | 2.3 | 2.3 | 2.3 |
| | (C) | Polyacrylic acid | 1.3 | 1.3 | 1.3 | 1.3 | 1.3 | 1.3 |
| | | amide copolymer 1 | | | | | | |

| | | | | | | | |
|---|---|---|---|---|---|---|---|
| | BG | 7.0 | 7.0 | 7.0 | 7.0 | 7.0 | 7.0 |
| | DPG | 10.0 | 10.0 | 10.0 | 10.0 | 10.0 | 10.0 |
| | Glycerin | 8.5 | 8.5 | 8.5 | 8.5 | 8.5 | 8.5 |
| | Hydrogenated lecithin | 0.1 | 0.1 | 0.1 | 0.1 | 0.1 | 0.1 |
| | PEG-60 hydrogenated | 0.3 | 0.3 | 0.3 | 0.3 | 0.3 | 0.3 |
| | castor oil | | | | | | |
| | Preservative | Adequate | Adequate | Adequate | Adequate | Adequate | Adequate |
| | | amount | amount | amount | amount | amount | amount |
| Oil phase | Neopentyl glycol | 1.1 | 1.1 | 1.1 | 1.1 | 1.1 | 1.1 |
| | diethylhexanoate | | | | | | |
| | Squalane | 0.5 | 0.5 | 0.5 | 0.5 | 0.5 | 0.5 |
| | Defoamer | Adequate | Adequate | Adequate | Adequate | Adequate | Adequate |
| | | amount | amount | amount | amount | amount | amount |
| | Antioxidant | Adequate | Adequate | Adequate | Adequate | Adequate | Adequate |
| | | amount | amount | amount | amount | amount | amount |
| Evaluation | Hardness (N) | 0.47 | 0.40 | 0.40 | 0.41 | 0.54 | 0.43 |
| | Durability (score) | B (3.0) | A (4.5) | A (5.0) | A (5.0) | A (5.0) | D (1.0) |
| | Usability | B | B | A | B | B | A |

| | | | Comparative | Comparative | Comparative | Comparative |
|---|---|---|---|---|---|---|
| | | | Example 4 | Example 5 | Example 6 | Example 7 |
| Aqueous | Purified water | | to 100 | to 100 | to 100 | to 100 |
| phase | (A-1) | Natural mica 1 | — | — | — | — |
| | | Natural mica 2 | — | — | — | — |
| | | Mica titanium | — | — | — | — |
| | | Titanium | — | — | — | — |
| | | oxide-coated | | | | |
| | | glass powder 1 | | | | |
| | | Titanium | — | — | — | — |
| | | oxide-coated | | | | |
| | | glass powder 2 | | | | |

| Natural mica 3 | | 30.0 | | — | — |
|---|---|---|---|---|---|
| Natural mica 4 | | — | 10.5 | — | — |
| Talc | | — | — | 10.5 | — |
| Spherical silica | | — | — | — | 10.5 |
| Colorant | | 1.0 | 1.0 | 1.0 | 1.0 |
| (B) | Agar | 2.3 | 2.3 | 2.3 | 2.3 |
| (C) | Polyacrylic acid | 1.3 | 1.3 | 1.3 | 1.3 |
| | amide copolymer 1 | | | | |

| | | | | | |
|---|---|---|---|---|---|
| | BG | 7.0 | 7.0 | 7.0 | 7.0 |
| | DPG | 10.0 | 10.0 | 10.0 | 10.0 |
| | Glycerin | 8.5 | 8.5 | 8.5 | 8.5 |
| | Hydrogenated lecithin | 0.1 | 0.1 | 0.1 | 0.1 |
| | PEG-60 hydrogenated | 0.3 | 0.3 | 0.3 | 0.3 |
| | castor oil | | | | |
| | Preservative | Adequate | Adequate | Adequate | Adequate |
| | | amount | amount | amount | amount |
| Oil phase | Neopentyl glycol | 1.1 | 1.1 | 1.1 | 1.1 |
| | diethylhexanoate | | | | |
| | Squalane | 0.5 | 0.5 | 0.5 | 0.5 |
| | Defoamer | Adequate | Adequate | Adequate | Adequate |
| | | amount | amount | amount | amount |
| | Antioxidant | Adequate | Adequate | Adequate | Adequate |
| | | amount | amount | amount | amount |
| Evaluation | Hardness (N) | 0.74 | 0.43 | 0.40 | 0.40 |
| | Durability (score) | A (5.0) | D (1.3) | D (1.5) | D (1.5) |
| | Usability | D | A | C | D |

TABLE 3

| | | | Comparative | Example | Example | Example | Example | Example | Example | Example |
|---|---|---|---|---|---|---|---|---|---|---|
| | | | Example 8 | 13 | 14 | 15 | 16 | 17 | 18 | 19 |
| Aqueous | Purified water | | to 100 | to 100 | to 100 | to 100 | to 100 | to 100 | to 100 | to 100 |
| phase | (A-1) | Synthetic | — | — | — | — | — | — | | — |
| | | fluorphlogopite 1 | | | | | | | | |
| | | Synthetic | — | — | — | — | — | — | — | — |
| | | fluorphlogopite 2 | | | | | | | | |
| | | Synthetic | 2.0 | 3.5 | 5.0 | 7.0 | 10.0 | 15.0 | 20.0 | 30.0 |
| | | fluorphlogopite 3 | | | | | | | | |
| | Colorant | | 1.0 | 1.0 | 1.0 | 1.0 | 1.0 | 1.0 | 1.0 | 1.0 |
| | (B) | Agar | 2.3 | 2.3 | 2.3 | 2.3 | 2.3 | 2.3 | 2.3 | 2.3 |
| | (C) | Polyacrylic acid | 1.3 | 1.3 | 1.3 | 1.3 | 1.3 | 1.3 | 1.3 | 1.3 |
| | | amide copolymer 1 | | | | | | | | |

| | | | | | | | | | |
|---|---|---|---|---|---|---|---|---|---|
| | BG | 7.0 | 7.0 | 7.0 | 7.0 | 7.0 | 7.0 | 7.0 | 7.0 |
| | DPG | 10.0 | 10.0 | 10.0 | 10.0 | 10.0 | 10.0 | 10.0 | 10.0 |
| | Glycerin | 8.5 | 8.5 | 8.5 | 8.5 | 8.5 | 8.5 | 8.5 | 8.5 |
| | Hydrogenated lecithin | 0.1 | 0.1 | 0.1 | 0.1 | 0.1 | 0.1 | 0.1 | 0.1 |
| | PEG-60 hydrogenated castor oil | 0.3 | 0.3 | 0.3 | 0.3 | 0.3 | 0.3 | 0.3 | 0.3 |
| | Preservative | Adequate | Adequate | Adequate | Adequate | Adequate | Adequate | Adequate | Adequate |
| | | amount | amount | amount | amount | amount | amount | amount | amount |
| Oil phase | Neopentyl glycol | 1.1 | 1.1 | 1.1 | 1.1 | 1.1 | 1.1 | 1.1 | 1.1 |
| | diethylhexanoate | | | | | | | | |
| | Squalane | 0.5 | 0.5 | 0.5 | 0.5 | 0.5 | 0.5 | 0.5 | 0.5 |
| | Defoamer | Adequate | Adequate | Adequate | Adequate | Adequate | Adequate | Adequate | Adequate |
| | | amount | amount | amount | amount | amount | amount | amount | amount |
| | Antioxidant | Adequate | Adequate | Adequate | Adequate | Adequate | Adequate | Adequate | Adequate |
| | | amount | amount | amount | amount | amount | amount | amount | amount |
| Evaluation | Hardness (N) | 0.28 | 0.28 | 0.35 | 0.32 | 0.41 | 0.54 | 1.17 | 0.97 |
| | Durability (score) | D (1.0) | C (2.3) | B (3.8) | B (3.8) | A (5.0) | A (5.0) | A (5.0) | A (5.0) |
| | Usability | B | B | A | A | A | B | B | C |

TABLE 4

| | | | Example | Example | Example | Example | Example | Comparative | Comparative |
|---|---|---|---|---|---|---|---|---|---|
| | | | 20 | 21 | 22 | 23 | 24 | Example 9 | Example 10 |
| Aqueous | Purified water | | to 100 | to 100 | to 100 | to 100 | to 100 | to 100 | to 100 |
| phase | (A-1) | Synthetic | — | — | — | — | — | — | — |
| | | fluorphlogopite 1 | | | | | | | |
| | | Synthetic | — | — | — | — | — | — | — |
| | | fluorphlogopite 2 | | | | | | | |
| | | Synthetic | 20.0 | 10.0 | 20.0 | 10.0 | 10.0 | 10.0 | 20.0 |
| | | fluorphlogopite 3 | | | | | | | |
| | Colorant | | 1.0 | 1.0 | 1.0 | 1.0 | 1.0 | 1.0 | 1.0 |
| | (B) | Agar | 1.0 | 3.5 | 2.3 | 1.8 | — | — | — |
| | | Gellan gum | — | — | — | — | 2.3 | — | — |
| | (C) | carrageenan | — | — | — | — | — | 2.3 | — |
| | | Locust bean gum | — | — | — | — | — | — | 1.4 |
| | | Polyacrylic acid | 1.3 | 1.3 | 0.5 | 2.0 | 1.3 | 1.3 | 1.3 |
| | | amide copolymer 1 | | | | | | | |

| | | | | | | | | |
|---|---|---|---|---|---|---|---|---|
| | BG | 7.0 | 7.0 | 7.0 | 7.0 | 7.0 | 7.0 | 7.0 |
| | DPG | 10.0 | 10.0 | 10.0 | 10.0 | 10.0 | 10.0 | 10.0 |
| | Glycerin | 8.5 | 8.5 | 8.5 | 8.5 | 8.5 | 8.5 | 8.5 |
| | Hydrogenated lecithin | 0.1 | 0.1 | 0.1 | 0.1 | 0.1 | 0.1 | 0.1 |
| | PEG-60 hydrogenated castor oil | 0.3 | 0.3 | 0.3 | 0.3 | 0.3 | 0.3 | 0.3 |
| | Preservative | Adequate | Adequate | Adequate | Adequate | Adequate | Adequate | Adequate |
| | | amount | amount | amount | amount | amount | amount | amount |
| Oil phase | Neopentyl glycol | 1.1 | 1.1 | 1.1 | 1.1 | 1.1 | 1.1 | 1.1 |
| | diethylhexanoate | | | | | | | |
| | Squalane | 0.5 | 0.5 | 0.5 | 0.5 | 0.5 | 0.5 | 0.5 |
| | Defoamer | Adequate | Adequate | Adequate | Adequate | Adequate | Adequate | Adequate |
| | | amount | amount | amount | amount | amount | amount | amount |
| | Antioxidant | Adequate | Adequate | Adequate | Adequate | Adequate | Adequate | Adequate |
| | | amount | amount | amount | amount | amount | amount | amount |
| Evaluation | Hardness (N) | 0.58 | 1.12 | 0.48 | 0.41 | 1.24 | 0.13 | 0.05 |
| | Durability (score) | B (3.8) | B (3.8) | B (3.5) | B (3.5) | A (5.0) | D (1.0) | D (1.0) |
| | Usability | B | C | B | B | C | C | D |

In Tables 1 to 4, details of respective raw materials are as follows.

<Powder Component>

Synthetic fluorphlogopite 1: “PDM-10L” (product name, manufactured by Topy Industries, Ltd., average particle size: 12 μm, aspect ratio: 60)

Synthetic fluorphlogopite 2: “PDM-20L” (product name, manufactured by Topy Industries, Ltd., average particle size: 20 μm, aspect ratio: 70)

Synthetic fluorphlogopite 3: “PDM-40L” (product name, manufactured by Topy Industries, Ltd., average particle size: 40 m, aspect ratio: 80)

Synthetic fluorphlogopite 4: “PDM-10S” (product name, manufactured by Topy Industries, Ltd., average particle size: 12 μm, aspect ratio: 25)

Synthetic fluorphlogopite 5: “PDM-5L” (product name, manufactured by Topy Industries, Ltd., average particle size: 7 μm, aspect ratio: 40)

Natural mica 1: “MICA #2000” (product name, manufactured by Wakita Kogyo, Ltd., average particle size: 27.5 μm, aspect ratio: 42.5)

Natural mica 2: “MICA POWDER SA-350” (product name, manufactured by YAMAGUCHI MICA, Co., Ltd., average particle size: 42 μm, aspect ratio: 80)

Natural mica 3: “Serisite LQ-15” (product name, manufactured by Ohchem Commerce Co., Ltd., average particle size: 9 μm, aspect ratio: 22)

Natural mica 4: “Serisite FSE” (product name, manufactured by Sanshin Mining Ind. Co., Ltd., average particle size: 7 μm, aspect ratio: 40)

Mica titanium 1: “Cosmetica Glitter White 9000E” (product name, manufactured by CQV Co., Ltd., average particle size: 45 to 50 μm, aspect ratio: 145)

Titanium oxide-coated glass powder 1: “Microglas Metashine MT1080RS” (product name, manufactured by Nippon Sheet Glass Co., Ltd., average particle size: 80 μm, aspect ratio: 80)

Titanium oxide-coated glass powder 2: “Microglas Metashine MT1120RS” (product name, manufactured by Nippon Sheet Glass Co., Ltd., average particle size: 120 μm, aspect ratio: 120)

Talc: “Talc MS-KY” (product name, manufactured by Nippon Talc Co., Ltd., average particle size: 23 μm, aspect ratio: 25)

Spherical silica: “SILICA MICRO BEAD BA-” (product name, manufactured by JGC C&C, average particle size: 16 μm)

Colorant: Iron oxide, titanium oxide, and Red 226

<Water-Soluble Thickener>

Polyacrylic acid amide copolymer 1: “SEPINOV EMT 10” (product name, manufactured by SEPPIC) (mixture containing 90% by mass of hydroxyethyl acrylate/sodium acryloyldimethyl taurate copolymer)

### Example 25: Blusher

<Aqueous Phase>

| | (Blending proportion |
|---|---|
| (Component) | (% by mass)) |
| Purified water | remainder |
| (A-1) Synthetic fluorphlogopite 3 | 11.0 |
| (A-1) Synthetic fluorphlogopite 1 | 2.0 |
| Red 226 | 0.5 |
| Yellow iron oxide | 0.9 |
| Red iron oxide | 0.2 |
| Titanium oxide | 0.1 |
| (B) Agar | 2.3 |
| (C) Polyacrylic acid amide copolymer 2 | 3.0 |
| (C) Xanthan gum | 0.1 |
| BG | 7.5 |
| DPG | 9.0 |
| Glycerin | 8.5 |
| Hydrogenated lecithin | 0.1 |
| PEG-60 hydrogenated castor oil | 0.3 |
| Preservative | adequate amount |

<Oil Phase>

| | (Blending proportion |
|---|---|
| (Component) | (% by mass)) |
| Cetyl ethylhexanoate | 1.5 |
| Defoamer | adequate amount |
| Antioxidant | adequate amount |

Details of the above-mentioned components are the same as those already described above except the component shown below.

Polyacrylic acid amide copolymer 2: Product name “SIMULGEL EG” (manufactured by SEPPIC) (mixture containing 40% by mass of sodium acrylate/sodium acryloyldimethyl taurate copolymer)

<Production Method>

First, aqueous phase components were dispersed in purified water, and this dispersion was heated and dissolved at 90° C. or higher, to prepare an aqueous phase portion. Subsequently, to the aqueous phase portion was added an oil phase portion, in which oil phase components were uniformly mixed, and the mixture was sufficiently stirred and emulsified. The obtained solution was charged into a predetermined mold at 60° C. or higher and cooled to obtain an oil-in-water type solid cosmetic material having a shape similar to that illustrated in FIG. 1. Then, the obtained solid cosmetic material was transferred into a predetermined container that causes a convex portion of the oil-in-water type solid cosmetic material to be exposed, and this was used as a sample.

<Evaluation>

For the obtained blusher, evaluation was performed in the same manner as above. As a result, the blusher was evaluated as having a hardness of 0.4 N, durability of “A (4.2)”, and usability of “B”.

### Example 26: Highlighter

<Aqueous Phase>

| | (Blending proportion |
|---|---|
| (Component) | (% by mass)) |
| Purified water | remainder |
| (A-1) Synthetic fluorphlogopite 3 | 9.0 |
| (A-1) Mica titanium 1 | 3.0 |
| Mica titanium | 2.0 |
| Titanium oxide | 0.2 |
| (B) Agar | 2.0 |
| (C) Cellulose gum | 1.0 |
| (C) Xanthan gum | 0.1 |
| (B) Gellan gum | 0.1 |
| BG | 10.0 |
| DPG | 6.0 |
| Glycerin | 8.0 |
| Hydrogenated lecithin | 0.1 |
| PEG-60 hydrogenated castor oil | 0.3 |
| Preservative | adequate amount |

<Oil Phase>

| | (Blending proportion |
|---|---|
| (Component) | (% by mass)) |
| Neopentyl glycol diethylhexanoate | 1.0 |
| Squalane | 0.5 |
| Defoamer | adequate amount |
| Antioxidant | adequate amount |

Details of the above-mentioned components are the same as those already described above.

<Production Method>

First, aqueous phase components were dispersed in purified water, and this dispersion was heated and dissolved at 90° C. or higher, to prepare an aqueous phase portion. Subsequently, to the aqueous phase portion was added an oil phase portion, in which oil phase components were uniformly mixed, and the mixture was sufficiently stirred and emulsified. The obtained solution was charged into a predetermined mold at 60° C. or higher and cooled to obtain an oil-in-water type solid cosmetic material having a shape similar to that illustrated in FIG. 1. Then, the obtained solid cosmetic material was transferred into a predetermined container that causes a convex portion of the oil-in-water type solid cosmetic material to be exposed, and this was used as a sample.

<Evaluation>

For the obtained highlighter, evaluation was performed in the same manner as above. As a result, the highlighter was evaluated as having a hardness of 0.38 N, durability of “A (4.5)”, and usability of “A”.

### Example 27: Foundation

<Aqueous Phase>

| | (Blending proportion |
|---|---|
| (Component) | (% by mass)) |
| Purified water | remainder |
| (A-1) Synthetic fluorphlogopite 3 *1 | 5.0 |
| Surface-treated titanium oxide *1 | 11.0 |
| Surface-treated yellow iron oxide *1 | 2.0 |
| Surface-treated red iron oxide *1 | 0.3 |
| (B) Agar | 2.0 |
| (C) Polyacrylic acid amide copolymer 1 | 1.0 |
| (B) Gellan gum | 0.2 |
| BG | 10.0 |
| DPG | 4.0 |
| Glycerin | 11.0 |
| Hydrogenated lecithin | 0.5 |
| Preservative | adequate amount |

<Oil Phase>

| | (Blending proportion |
|---|---|
| (Component) | (% by mass)) |
| Ethylhexyl methoxycinnamate | 6.0 |
| Octocrylene | 3.0 |
| Diphenylsiloxy phenyl trimethicone | 3.0 |
| Defoamer | adequate amount |
| Antioxidant | adequate amount |

Details of the above-mentioned components are the same as those already described above.

However, regarding *1, treatment was performed using 3 parts by mass of alkylsilane and 3 parts by mass of hydrogenated lecithin with respect to 100 parts by mass of the powder.

<Production Method>

First, aqueous phase components were dispersed in purified water, and this dispersion was heated and dissolved at 90° C. or higher, to prepare an aqueous phase portion. Subsequently, to the aqueous phase portion was added an oil phase portion, in which oil phase components were uniformly mixed, and the mixture was sufficiently stirred and emulsified. The obtained solution was charged into a predetermined mold at 60° C. or higher and cooled to obtain an oil-in-water type solid cosmetic material having a shape similar to that illustrated in FIG. 1. Then, the obtained solid cosmetic material was transferred into a predetermined container that causes a convex portion of the oil-in-water type solid cosmetic material to be exposed, and this was used as a sample.

<Evaluation>

For the obtained foundation, evaluation was performed in the same manner as above. As a result, the foundation was evaluated as having a hardness of 0.4 N, durability of “B (3.5)”, and usability of “B”.

### Example 28: Eye Shadow

<Aqueous Phase>

| | (Blending proportion |
|---|---|
| (Component) | (% by mass)) |
| Purified water | remainder |
| (A-1) Synthetic fluorphlogopite 3 | 10.0 |
| (A-1) Mica titanium 2 | 4.5 |
| (A-1) Red iron oxide-coated mica titanium 1 | 1.5 |
| (B) Agar | 2.3 |
| (C) Polyacrylic acid amide copolymer 1 | 1.2 |
| (B) Gellan gum | 0.1 |
| (C) Xanthan gum | 0.1 |
| BG | 6.8 |
| DPG | 9.0 |
| Glycerin | 8.5 |
| Hydrogenated lecithin | 0.1 |
| PEG-60 hydrogenated castor oil | 0.3 |
| Preservative | adequate amount |

<Oil Phase>

| | (Blending proportion |
|---|---|
| (Component) | (% by mass)) |
| Neopentyl glycol diethylhexanoate | 1.1 |
| Squalane | 0.5 |
| Defoamer | adequate amount |
| Antioxidant | adequate amount |

Details of the above-mention components are the same as those already described above except the components shown below.

Mica titanium 2: “FLAMENCO SPARKLE GOLD” (product name, manufactured by Merck & Co., Inc., average particle size: 44 μm, aspect ratio: 94)

Red iron oxide-coated mica titanium 1: “Colorona Sienna” (product name, manufactured by BASF, average particle size: 22 μm, aspect ratio: 75)

<Production Method>

First, aqueous phase components were dispersed in purified water, and this dispersion was heated and dissolved at 90° C. or higher, to prepare an aqueous phase portion. Subsequently, to the aqueous phase portion was added an oil phase portion, in which oil phase components were uniformly mixed, and the mixture was sufficiently stirred and emulsified. The obtained solution was charged into a predetermined mold at 60° C. or higher and cooled to obtain an oil-in-water type solid cosmetic material having a shape similar to that illustrated in FIG. 1. Then, the obtained solid cosmetic material was transferred into a predetermined container that causes a convex portion of the oil-in-water type solid cosmetic material to be exposed, and this was used as a sample.

<Evaluation>

For the obtained eye shadow, evaluation was performed in the same manner as above. As a result, the eye shadow was evaluated as having a hardness of 0.55 N, durability of “A (5.0)”, and usability of “B”.

### Example 29: Stick-Shaped Blusher

<Aqueous Phase>

| | (Blending proportion |
|---|---|
| (Component) | (% by mass)) |
| Purified water | remainder |
| (A-1) Synthetic fluorphlogopite 3 | 12.0 |
| Titanium oxide | 1.5 |
| Red iron oxide | 0.4 |
| (B) Agar | 2.6 |
| (C) Polyacrylic acid amide copolymer 1 | 1.2 |
| (B) Gellan gum | 0.1 |
| (C) Xanthan gum | 0.1 |
| BG | 10.8 |
| DPG | 6.0 |
| Glycerin | 8.5 |
| Hydrogenated lecithin | 0.1 |
| PEG-60 hydrogenated castor oil | 0.3 |
| Preservative | adequate amount |

<Oil Phase>

| | (Blending proportion |
|---|---|
| (Component) | (% by mass)) |
| Neopentyl glycol diethylhexanoate | 1.6 |
| Defoamer | adequate amount |
| Antioxidant | adequate amount |

<Production Method>

First, aqueous phase components were dispersed in purified water, and this dispersion was heated and dissolved at 90° C. or higher, to prepare an aqueous phase portion. Subsequently, to the aqueous phase portion was added an oil phase portion, in which oil phase components were uniformly mixed, and the mixture was sufficiently stirred and emulsified. The obtained solution was charged into a predetermined mold at 60° C. or higher and cooled to obtain an oil-in-water type solid cosmetic material having a stick shape. Then, the obtained solid cosmetic material was transferred into a predetermined container to which the oil-in-water type solid cosmetic material is delivered, and this was used as a sample.

<Evaluation>

For the obtained stick-shaped blusher, evaluation was performed in the same manner as above. As a result, the stick-shaped blusher was evaluated as having a hardness of 0.75 N, durability of “B (3.2)”, and usability of “B”.

### REFERENCE SIGNS LIST

1 Container
2 Oil-in-water type solid cosmetic material
3 Container main body
3d Holding member
3e Annular holding member
3e1, 3e2, 3e3 Upper surface of annular holding member
3f Radial holding member
3g Charging port (opening)
4 Top lid
5 Bottom lid
6 Lipstick mold (mold)
6a Concave portion of lipstick mold
7 Holding body
8 Charging nozzle
9 Oil-in-water type solid cosmetic material in dissolved state
10 Molding space

What is claimed is:

1. An oil-in-water type solid cosmetic material, comprising:
a powder component;
a gelling agent;
a water-soluble thickener;
water; and
an oiling agent,
wherein a plate-like powder, which has an average particle size of 10 to 200 μm and has an aspect ratio of 40 to 200, is contained, as the powder component, in an amount of 3% to 30% by mass based on a total amount of the cosmetic material; and
agar and/or gellan gum is contained as the gelling agent.

2. The oil-in-water type solid cosmetic material according to claim 1,
wherein the plate-like powder is at least one powder selected from the group consisting of synthetic fluorphlogopite, natural mica, metal oxide-coated mica, and metal oxide-coated glass powder.

3. The oil-in-water type solid cosmetic material according to claim 1,
wherein at least one selected from the group consisting of polyacrylic acid amide copolymers, xanthan gums, and cellulose derivatives is contained, as the water-soluble thickener, in an amount of 0.3% to 2.5% by mass based on a total amount of the cosmetic material.

4. The oil-in-water type solid cosmetic material according to claim 1,
wherein a sum of contents of the agar and the gellan gum is 0.5% to 4% by mass based on a total amount of the cosmetic material.

5. The oil-in-water type solid cosmetic material according to claim 1,
wherein a content of the water is equal to or higher than a content of the powder component.

6. The oil-in-water type solid cosmetic material according to claim 1,
wherein the oil-in-water type solid cosmetic material is molded three-dimensionally to be applied to skin by direct contact.

7. The oil-in-water type solid cosmetic material according to claim 1,
wherein the oil-in-water type solid cosmetic material is a molded body for being applied to skin by direct contact.

8. The oil-in-water type solid cosmetic material according to claim 7,
wherein the molded body has a convex portion having a dome shape, a pyramid shape, a diamond cut shape, or a columnar shape.

9. A cosmetic product, comprising:
a container; and
the oil-in-water type solid cosmetic material according to claim 1 which is accommodated in the container,
wherein the container has a container main body for holding the oil-in-water type solid cosmetic material, and the oil-in-water type solid cosmetic material is held in the container main body in a state of protruding therefrom; or
the container has a container main body for receiving the oil-in-water type solid cosmetic material, and the container main body has a holding member that holds the oil-in-water type solid cosmetic material and allows the oil-in-water type solid cosmetic material to protrude from the container main body.

10. The cosmetic product according to claim 9,
wherein the plate-like powder is at least one powder selected from the group consisting of synthetic fluorphlogopite, natural mica, metal oxide-coated mica, and metal oxide-coated glass powder.

11. The cosmetic product according to claim 9,
wherein the oil-in-water type solid cosmetic material contains at least one selected from the group consisting of polyacrylic acid amide copolymers, xanthan gums, and cellulose derivatives, as the water-soluble thickener, in an amount of 0.3% to 2.5% by mass based on a total amount of the cosmetic material.

12. The cosmetic product according to claim 9,
wherein a sum of contents of the agar and the gellan gum in the oil-in-water type solid cosmetic material is 0.5% to 4% by mass based on a total amount of the cosmetic material.

13. The cosmetic product according to claim 9,
wherein a content of the water in the oil-in-water type solid cosmetic material is equal to or higher than a content of the powder component.

14. The cosmetic product according to claim 9,
wherein the oil-in-water type solid cosmetic material is molded three-dimensionally to be applied to skin by direct contact.

15. The cosmetic product according to claim 9,
wherein the oil-in-water type solid cosmetic material is a molded body for being applied to skin by direct contact.

16. The cosmetic product according to claim 15,
wherein the molded body has a convex portion having a dome shape, a pyramid shape, a diamond cut shape, or a columnar shape.
